(19) United States
(12) Patent Application Publication
(10) Pub. No.: US 2023/0063836 A1
(43) Pub. Date: Mar. 2, 2023

(54) METHODS FOR DETERMINING RESTRICTION SCHEMES IN SMART CITIES, INTERNET OF THINGS SYSTEMS, AND MEDIUM THEREOF

(71) Applicant: CHENGDU QINCHUAN IOT TECHNOLOGY CO., LTD., Chengdu (CN)

(72) Inventors: Zehua SHAO, Chengdu (CN); Bin LIU, Chengdu (CN); Yaqiang QUAN, Chengdu (CN); Yong LI, Chengdu (CN); Xiaojun WEI, Chengdu (CN)

(73) Assignee: CHENGDU QINCHUAN IOT TECHNOLOGY CO., LTD., Chengdu (CN)

(21) Appl. No.: 18/052,931

(22) Filed: Nov. 6, 2022

(30) Foreign Application Priority Data
Oct. 13, 2022 (CN) 202211253059.2

(51) Int. Cl. G06Q 50/26; G01N 33/00

(52) U.S. Cl. CPC G06Q 50/26; G01N 33/0075

(57) ABSTRACT

The disclosure provides a method for determining a restriction scheme in a smart city, which is implemented based on an Internet of Things system for determining the restriction scheme in the smart city. The method includes: determining the urban pollution information through an object platform, sending the urban pollution information to a management platform through a sensor network platform, determining the optimal restriction scheme of the city according to the urban pollution information through the management platform, and sending the optimal restriction scheme to the user platform through the service platform. The urban pollution information including at least one of urban air image, air quality information, and vehicle exhaust information.

### CROSS-REFERENCE TO RELATED DISCLOSURES

This application claims priority to Chinese Patent Application No. 202211253059.2, filed on Oct. 13, 2022, the entire contents of which are hereby incorporated by reference.

### TECHNICAL FIELD

The present disclosure relates to the field of urban planning, and in particular to a method for determining a restriction scheme in a smart city, an Internet of Things system, and a medium.

### BACKGROUND

With the continuous development of the social economy, the number of vehicles is also increasing. The ever-increasing number of vehicles has brought serious congestion problems to urban traffic, and the exhaust gas emitted by vehicles has also caused considerable damage to the urban environment.

Therefore, it is necessary to provide a method for determining a restriction scheme in a smart city, Internet of Things system, and medium, so as to reduce exhaust emissions while alleviating urban congestion, thereby improving the urban environment.

### SUMMARY

One of the embodiments of this present disclosure provides a method for determining a restriction scheme in a smart city. The method includes: determining urban pollution information of a city through the object platform, and sending the urban pollution information to the management platform through the sensor network platform, the urban pollution information including at least one of urban air image, air quality information, and vehicle exhaust information; determining an optimal restriction scheme of the city according to the urban pollution information through the management platform, and sending the optimal restriction scheme to the user platform through the service platform, including: determining an urban pollution degree of the city according to the urban pollution information; determining at least one candidate restriction region based on the pollution degree of the city; determining the candidate restriction level of at least one candidate restriction region, and generating a plurality of initial restriction schemes; determining the optimal restriction scheme through processing the plurality of initial restriction schemes based on the preset algorithm; and sending the optimal restriction scheme to a service platform and forwarding the optimal restriction scheme to the user platform.

One of the embodiments of the present disclosure provides an Internet of Things system for determining the restriction scheme in the smart city. The system includes a user platform, a service platform, a management platform, an object platform, and a sensor network platform. The object platform is configured to determine urban pollution information of a city, and send the urban pollution information to the management platform through the sensor network platform. The urban pollution information includes at least one of urban air image, air quality information, and vehicle exhaust information. The management platform is configured to determine the optimal restriction scheme of the city according to the urban pollution information, and send the optimal restriction scheme to the user platform through the service platform. To determine an optimal restriction scheme of the city according to the urban pollution information and send the optimal restriction scheme to the user platform through the service platform, the management platform is further configured to determine the urban pollution degree of the city according to the urban pollution information; determine at least one candidate restriction region based on the urban pollution degree of the city; determine the candidate restriction level of at least one candidate restriction region, and generate a plurality of initial restriction schemes; determine the optimal restriction scheme through processing the plurality of initial restriction schemes based on the preset algorithm; and send the optimal restriction scheme to the service platform and forwarding the optimal restriction scheme to the user platform.

One of the embodiments of the present disclosure provides a non-transitory computer-readable storage medium, the storage medium stores computer instructions, and after the computer reads the computer instructions in the storage medium, the computer executes the above method for determining a restriction scheme in a smart city.

### BRIEF DESCRIPTION OF THE DRAWINGS

The present disclosure is further described in terms of exemplary embodiments. These exemplary embodiments are described in detail with reference to the drawings. These embodiments are not limited. In these embodiments, the same number represents the same structure, wherein:

FIG. 1 is a schematic diagram of an Internet of Things system for determining a restriction scheme in a smart city according to some embodiments of the present disclosure;

FIG. 2 is an exemplary flowchart of a method for determining a restriction scheme in a smart city according to some embodiments of the present disclosure;

FIG. 3 is an exemplary flowchart of determining an optimal restriction scheme according to some embodiments of the present disclosure;

FIG. 4 is an exemplary structural diagram of an evaluation model according to some embodiments of the present disclosure;

FIG. 5 is an exemplary structural diagram of a pollution prediction model according to some embodiments of the present disclosure.

### DETAILED DESCRIPTION

In order to more clearly explain the technical scheme of the embodiment of the present disclosure, the accompanying drawings required in the description of the embodiment will be briefly introduced below. Obviously, the drawings in the following description are only some examples or embodiments of the disclosure. For those skilled in the art, the present disclosure may also be applied to other similar situations according to these drawings without paying creative labor. Unless obviously obtained from the context or the context illustrates, otherwise, the same numeral in the drawings refers to the same structure or operation.

It should be understood that the “system”, “device”, “unit” and/or “module” used herein is a method for distinguishing different components, elements, components, parts, or assemblies at different levels. However, the terms may be displaced by another expression if they achieve the same purpose.

As shown in the present disclosure and the claims, unless the context clearly suggests exceptional circumstances, the words “a”, “an” and/or “the” do not specifically refer to the singular, but may also include the plural. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts are used in the present disclosure to illustrate the operations performed by the system according to the embodiment of the present disclosure. It should be understood that the foregoing or following operations may be not necessarily performed exactly in order. Instead, the operations may be processed in reverse order or simultaneously. Moreover, one or more other operations may be added to the flowcharts or one or more operations may be removed from the flowcharts.

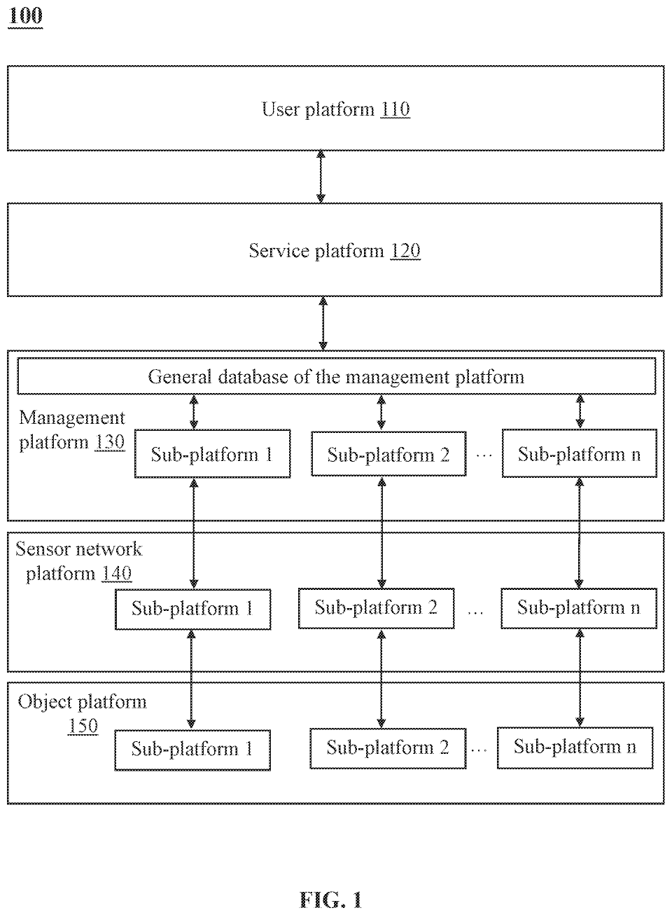

FIG. 1 is a schematic diagram of an Internet of Things system for determining a restriction scheme in a smart city according to some embodiments of the present disclosure.

In some embodiments, the Internet of Things system 100 for determining a restriction scheme in a smart city may be applied to a traffic management system of the city and used to execute a method for determining a traffic restriction scheme in a smart city. The city may be the execution object of the Internet of Things system 100 for determining the restriction scheme in the smart city, and the Internet of Thing system 100 for determining the restriction scheme in the smart city may determine the restriction scheme of the city according to relevant information of the city (such as traffic conditions, pollution conditions, etc.). For example, the Internet of Things system 100 for determining the restriction scheme in the smart city may determine the current restriction scheme of the city according to the air pollution situation of the city.

As shown in FIG. 1, the Internet of Things system 100 for determining a restriction scheme in a smart city may include: a user platform 110, a service platform 120, a management platform 130, a sensor network platform 140, and an object platform 150 that interact in sequence.

The user platform 110 is a user-oriented platform. In some embodiments, the user platform 110 is configured as a terminal device (e.g., a mobile phone, a tablet computer, etc.), which may feedback the vehicle restriction scheme of each region of the city to the user. For example, the user platform 110 may provide restriction information of street A to the user.

In some embodiments, the user platform 110 may interact downward with the service platform 120. For example, the user platform 110 may issue a query instruction of the vehicle restriction scheme of each region of the city to the service platform 120, and receive the vehicle restriction scheme of each region of the city uploaded by the service platform 120.

The service platform 120 refers to a platform that provides users with query services for vehicle restriction schemes of various regions of the city. In some embodiments, the service platform adopts a centralized layout. The centralized layout means that the reception, processing, and transmission of data or/and information are carried out by the platform in a unified manner.

In some embodiments, the service platform 120 may interact downward with the management platform 130. For example, the service platform may issue the query instruction of the vehicle restriction scheme of each region of the city to management platform 130, and receive the vehicle restriction scheme uploaded by management platform 130.

In some embodiments, the service platform may also interact upward with the user platform. For example, the service platform may receive the query instruction of the vehicle restriction scheme issued by the user platform 110, and upload the vehicle restriction scheme to the user platform 110, etc.

The management platform 130 is a platform for executing the method for determining a restriction scheme in a smart city. In some embodiments, in response to the user's query requirement, the management platform 130 may also be used to process the relevant monitoring data of various regions of the city uploaded by the sensor network platform, and determine the vehicle restriction scheme of each region of the city.

The relevant monitoring data of various regions of the city refers to the monitoring data of different roads in each region of the city, which may include data related to air pollution and data related to traffic flow. The data related to air pollution refers to the monitoring data of air quality (such as the content of SO₂, NO₂, PM₁₀, PM_{2.5}, and other pollutants), which may be obtained based on instruments such as an air quality detector. The data related to the traffic flow refers to the data related to the traffic flow on the road in each region, the vehicle exhaust emission situation in each region, the road congestion situation in each region, etc., which may be obtained based on the camera device, etc.

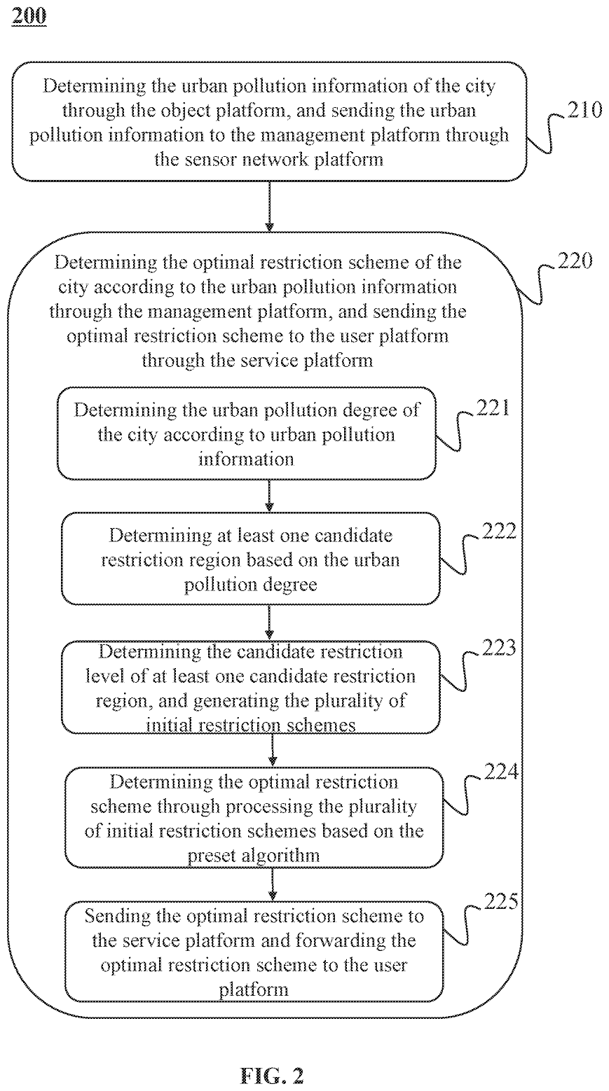

For more details about the management platform determining vehicle restriction schemes of various regions of the city based on the relevant monitoring data of various regions of the city, please refer to FIG. 2 and its related descriptions.

In some embodiments, the Internet of Things system for determining a restriction scheme in a smart city also includes a sensor network platform 140. The sensor network platform 140 is a platform for obtaining relevant monitoring data of various regions of the city. In some embodiments, the sensor network platform may be configured as a communication network and gateway.

In some embodiments, the sensor network platform 140 may interact downward with object platform 150. For example, the sensor network platform may receive relevant monitoring data uploaded by the object platform; and issue an instruction for obtaining relevant monitoring data to the object platform. In some embodiments, the sensor network platform 140 may also interact upward with the management platform 130. For example, the sensor network platform 140 may receive an instruction for obtaining relevant monitoring data issued by the management platform; and upload the relevant monitoring data to the management platform.

In some embodiments, the Internet of Things system for determining a restriction scheme in a smart city also includes an object platform 150. The object platform 150 is a platform for obtaining relevant monitoring data of various regions of the city, which may be deployed in different regions of the city. In some embodiments, the object platform is configured as a unique identification monitoring device, which may include camera device (for obtaining images of the region, such as air visibility, etc.), vehicle exhaust monitor (for obtaining exhaust emissions, etc.), air quality detector (for obtaining air pollution index, etc.), and other related equipment.

In some embodiments, the object platform 150 may interact upward with the sensor network platform 140. For example, the object platform may receive an instruction for obtaining relevant monitoring data issued by the sensor network platform; and upload the relevant monitoring data to the sensor network platform.

In some embodiments, the execution object (such as a city) of the Internet of Things system 100 for determining a traffic restriction scheme in a smart city is further divided (such as divided into multiple blocks) according to the actual needs, so as to determine a more accurate restriction scheme. For example, according to the urban planning of the city and the subordinate jurisdictions, the cities are divided into multiple regions (such as urban regions, blocks, etc.). In some embodiments, the Internet of Things system 100 for determining a restriction scheme in a smart city may be constructed distribute based on various regions.

In some embodiments, the management platform 130 may also be divided into a plurality of management sub-platforms according to urban regions. For example, the management platform may be divided into a plurality of different management sub-platforms according to administrative regions such as streets and communities of the city. In some embodiments, the management platform 130 may include a general database of the management platform and a plurality of management sub-platforms.

In some embodiments, the management platform 130 is configured as a second server in a combined front sub platform layout. The front sub-platform layout refers to that each sub-platform processes and manages the corresponding data, transmits the processed data to the general database, and then the general database uploads the processed data to other platforms after summarizing.

In some embodiments, the data interaction of the management platform includes that each management sub-platform receives relevant monitoring data of each region from the corresponding sensor network sub-platform; each management sub-platform processes and manages the relevant monitoring data of each region, for example, the relevant monitoring data of various roads in region A of city is uploaded to the vehicle management sub-platform of region A of city for management; each management sub-platform further processes the relevant monitoring data and uploads the processed data to the general database of the management platform; and the general database of the management platform uploads the summarized traffic-related data to the service platform, and the data uploaded to the service platform may also include the vehicle restriction schemes of various regions of the city.

In some embodiments, the management sub-platform processes the relevant monitoring data of different regions and then summarizes the relevant monitoring data into the general database, which may reduce the data processing pressure of the whole management platform and summarize the data of each independent management sub-platform for unified management.

In some embodiments, the sensor network platform 140 may also be divided into a plurality of sensor network sub-platforms according to the urban region. For example, the sensor network platform may be divided into a plurality of different sensor network sub-platforms according to the administrative region such as streets and communities of the city.

In some embodiments, the data interaction of the sensor network platform includes that the corresponding sub-platform processes and manages the relevant monitoring data, for example, data of the monitoring device deployed in the region A of city is uploaded to the sensor network sub-platform of region A of the city; and the sensor network sub-platform uploads the processed relevant monitoring data to the corresponding management sub-platform.

In some embodiments, the object platform 150 may also include a plurality of object sub-platforms. The plurality of object sub-platforms may respectively correspond to the plurality of different monitoring devices. The plurality of object sub-platforms may obtain relevant monitoring data and upload relevant monitoring data to corresponding sensor network sub-platforms.

In some embodiments, the Internet of Things system 100 for determining a traffic restriction scheme in a smart city may be used to execute the method for determining restriction scheme in the smart city, and the Internet of Things system for determining a restriction scheme in a smart city includes a user platform, a service platform, and a management platform. The object platform is configured to determine the urban pollution information of the city and send the urban pollution information to the management platform through the sensor network platform. The urban pollution information includes at least one of urban air image, air quality information and vehicle exhaust information. The management platform is configured to determine the optimal traffic restriction scheme of the city according to the urban pollution information, including following operations. The management platform is further configured to determine the urban pollution degree of the city according to the urban pollution information; determine at least one candidate restriction region according to the urban pollution degree; determine the candidate restriction level of at least one candidate restriction region, and generate a plurality of initial restriction schemes; and determine the optimal restriction scheme through processing the plurality of initial restriction schemes based on the preset algorithm. For more contents about the method for determining a restriction scheme in a smart city, please refer to FIG. 2 and its related descriptions.

In some embodiments, the object platform 150 is further configured to determine the urban pollution degree of the city through processing urban pollution information based on the pollution prediction model, and the pollution prediction model is a machine learning model. For more contents about determining the urban pollution degree, please refer to FIG. 2 and its related descriptions.

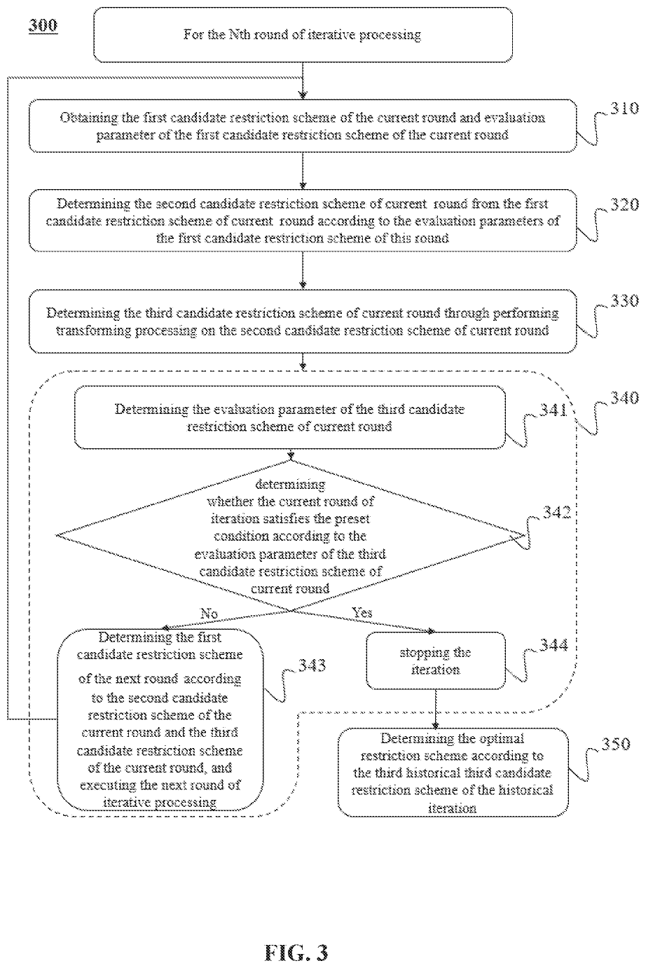

In some embodiments, the management platform 130 may also be configured to determine the optimal restriction scheme through performing at least one round of iterative processing on the plurality of initial restriction schemes based on the preset algorithm. For more contents about determining the optimal restriction scheme, please refer to FIG. 3 and its related descriptions.

Some embodiments of the present disclosure also provide a non-transitory computer readable storage medium, which stores computer instructions, and when the computer reads the computer instructions in the storage medium, the computer executes the method for determining a restriction scheme in a smart city.

It should be noted that the above descriptions of the Internet of Things system for determining restriction scheme in a smart city and its internal modules is only for convenience of description, and does not limit the present disclosure to the scope of the illustrated embodiments. It may be understood that after understanding the principle of the system, those skilled in the art may arbitrarily combine each module or form a subsystem to connect with other modules without departing from this principle. In some embodiments, the user platform 110, the service platform 120, the management platform 130, the sensor network platform 140 and the object platform 150 disclosed in FIG. 1 may be different modules in a system, or one module implementing the functions of two or more modules mentioned above. For example, each module may share one storage module, and each module may also have its own storage modules. Such deformations may be all within the scope of the protection of the present disclosure.

FIG. 2 is an exemplary flowchart of a method for determining a restriction scheme in a smart city according to some embodiments of the present disclosure. In some embodiments, process 200 may be executed by the Internet of Things system 100 for determining a restriction scheme in the smart city. As shown in FIG. 2, process 200 includes the following steps.

Step 210, determining the urban pollution information of the city through the object platform, and sending the urban pollution information to the management platform through the sensor network platform. In some embodiments, step 210 may be executed by the object platform 150.

Urban pollution information may be information that reflects urban pollution. The urban pollution may include pollution caused by vehicles (such as vehicle exhaust pollution, etc.). For example, urban pollution information may include at least one of urban air image, air quality information, and vehicle exhaust information.

Urban air image may refer to sky image of various regions in the city. The urban air image may reflect visible air pollution (such as smog, air color, etc.). In some embodiments, urban air image may be obtained by sensors (such as camera devices) preset at different position in the object platform.

Air quality information may refer to the meteorological data that reflect the air condition, quality, composition, or other information in the city. For example, air quality information may include the components and contents of pollutants such as PM_{2.5}, PM₁₀, nitrogen oxides, etc. in the air. In some embodiments, air quality information may be determined by meteorological institutions such as meteorological detection stations and meteorological detection points.

Vehicle exhaust information may be information that directly or indirectly reflects vehicle exhaust emissions in city. For example, vehicle exhaust information may include the number of vehicles on various roads in city and the corresponding vehicle exhaust emissions at present or in the future.

In some embodiments, the vehicle exhaust information may be determined by the sensor of the object platform. For example, the images of various roads in the city may be obtained by the camera devices preset at different positions in the object platform, and the number of vehicles on each road and exhaust emissions may be estimated according to the images. For example, the number of vehicles in the image may be determined by the target detection algorithm, and then the number of vehicles on each road in the city and the corresponding exhaust emissions may be estimated (such as regression analysis) based on the number of vehicles in the image.

In some embodiments, the object platform may package information in real-time or periodically after obtaining urban pollution information, and send the packaged information to the management platform through the sensor network platform.

Step 220, determining the optimal restriction scheme of the city according to the urban pollution information through the management platform, and sending the optimal restriction scheme to the user platform through the service platform. In some embodiments, step 220 may be executed by the management platform 130.

Restriction may refer to the control measures that restrict the entry, exit, or driving of vehicles in the city. For example, the restriction may include the restriction of license plate number, that is, vehicles whose license plate number meets the preset condition (for example, the tail number is a specific number or letter) cannot enter a specific region or street. For another example, the restriction may include a specific vehicle restriction, that is, vehicles with a specific vehicle type (such as heavy trucks) or other related attributes (such as vehicles with a displacement higher than 3.0) may be restricted from entering a specific region or street.

The restriction scheme may include information such as the specific content, execution time, and scope of applications of control measures of various restrictions in the city. For example, the current restriction scheme of city A may include that vehicle from other provinces cannot enter the second ring road of the city, and the local vehicles with tail numbers of 2 and 7 cannot enter the fifth ring road of the city from 8:00 to 16:00.

The optimal restriction scheme may refer to the optimal solution determined among feasible restriction schemes with the goal of minimizing the urban pollution caused by vehicles. That is, the optimal restriction scheme is a scheme that may minimize urban pollution among all the executable restriction schemes. In some embodiments, the optimal restriction scheme may also be the scheme with the lowest restriction cost among the restriction schemes that may achieve the preset purification target.

In some embodiments, the restriction scheme may be described by a restriction level. The restriction level may reflect the specific restriction degree of control measures of restriction. The higher the restriction level is, the higher the restriction degree of vehicles is. For example, level 0 may indicate no restriction; level 1 may indicate restriction of one tail number (such as tail number 3); level 2 may indicate restriction of two tail numbers (such as tail numbers 3 and 7); and level 3 may indicate restriction of three tail numbers (such as the tail numbers 3, 7, and 0). For another example, level 4 may indicate restriction of the passage of vehicles with exhaust emissions greater than a threshold (such as vehicles with exhaust emissions greater than 3.2 L). In some embodiments, considering that different restriction schemes may achieve the same or similar restriction effects, the same restriction level may include a plurality of restriction measures. For example, level of restriction of a vehicle with one tail number and level of restriction of a nonlocal vehicle may both be 1.

In some embodiments, the management platform 130 may determine the restriction level of each region according to the urban pollution information, so as to determine the optimal restriction scheme. The management platform 130 may send the determined optimal restriction scheme to the service platform, and the service platform may send the optimal restriction scheme to the user platform. The specific process is shown in FIG. 2, and step 220 may further include the following sub-steps.

Step 221, determining the urban pollution degree of the city according to urban pollution information.

The urban pollution degree may be used to quantitatively describe the pollution of urban air. For example, the urban pollution degree may be characterized by level. The higher the level is, the worse the pollution of the city is. In some embodiments, the urban pollution degree may also reflect the pollution of the vehicle to the city.

In some embodiments, the management platform may process the urban pollution information according to a preset rule to determine the urban pollution degree of the city. For example, the preset standard of urban pollution degree (such as the preset standards of various levels) may be compared with urban pollution information to determine the urban pollution degree. In some embodiments, the management platform may also determine the general pollution situation of the city and the pollution situation of non-vehicles according to the related information of road parts and the related information of non-road parts (such as suburbs and residential regions) in the urban pollution information, and then determine the pollution situation of vehicles to the city.

In some embodiments, the management sub-platforms of the management platform may process urban pollution information of each region of city, thereby determining the urban pollution degree of each region.

In some embodiments, the urban pollution degree of a city may be estimated based on a machine learning algorithm, that is, the pollution prediction model may process the urban pollution information to determine the urban pollution degree of the city. The pollution prediction model is a trained machine learning model.

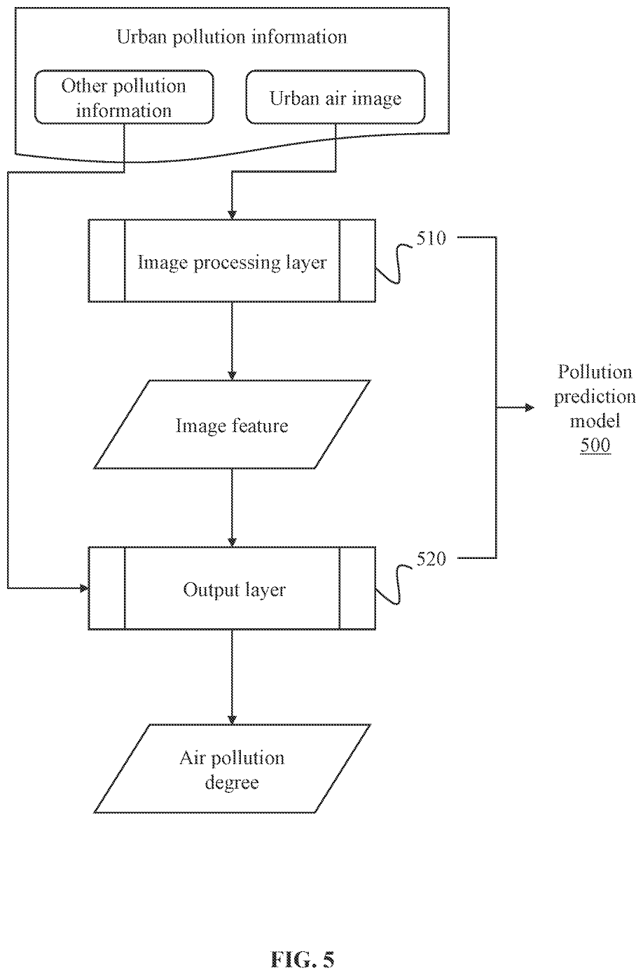

To further explain the data processing process of the pollution prediction model, FIG. 5 provides an exemplary structure diagram of the pollution prediction model.

As shown in FIG. 5, the pollution prediction model 500 may include an image processing layer 510 and an output layer 520. The image processing layer 510 may be Constitutional Neural Networks (CNN) model, and the output layer 520 may be Deep Neural Networks (DNN) model. In some embodiments, the urban air image in the urban pollution information may be input into the image processing layer 510 to determine the image feature of the urban air image. Then, the image feature and other pollution information (such as air quality information, vehicle exhaust information, etc.) in the urban pollution information may be input into the output layer 520 to determine the air pollution degree.

In some embodiments, the output of the image processing layer 510 may be the input of the output layer 520, and the image processing layer 510 and the output layer 520 may be obtained by jointly training.

In some embodiments, the sample data of the joint training includes historical pollution information of cities or regions, and the label may be the air pollution degree manually marked based on the corresponding historical pollution information. The image information in the historical pollution information may be input into the image processing layer 510 to obtain the image feature output by the image processing layer 510. The image feature used as training sample data and other information in the historical pollution information are input into the output layer 520 to obtain the air pollution degree output by the output layer 520. The loss function is constructed based on the manually marked air pollution degree and the air pollution degree output by the output layer, and the parameters of the image processing layer 510 and the output layer 520 are updated synchronously. Through parameter updating, the trained image processing layer 510 and the trained output layer 520 are obtained.

Step 222, determining at least one candidate restriction region based on the urban pollution degree.

Restriction region may refer to urban region where restriction measures are implemented. Candidate restriction region may refer to the candidate region where restriction measures are likely to be implemented, which may be determined by the management platform according to the urban pollution degree. For example, the candidate restriction region may be a district of the city, a main road of the city, a ring road of the city, etc.

In some embodiments, it may be determined whether a region is a candidate restriction region according to the urban pollution degree of each region. For example, when the urban pollution degree of each region is higher than a pollution degree threshold, the corresponding region may be taken as a candidate restriction region.

In some embodiments, the candidate restriction region may be determined based on other related data (such as traffic flow data). For example, the region where the traffic flow data is higher than the threshold may be determined as the initial candidate traffic restriction scheme based on the traffic flow data obtained by a third-party platform (such as the transportation department).

Step 223, determining the candidate restriction level of at least one candidate restriction region, and generating the plurality of initial restriction schemes.

The candidate restriction level may refer to the level of restriction measures planned to be implemented in the candidate restriction region. The candidate restriction level may be determined according to the urban pollution degree in the candidate restriction region. For example, the higher the urban pollution degree is, the higher the candidate restriction level is. The higher the candidate restriction level is, the stricter the restriction of vehicles in the city is.

In some embodiments, there may be a corresponding relationship between each candidate restriction level and the urban pollution degree. For example, if the urban pollution degree is less than 3, the candidate restriction level may be 1. If the urban pollution degree is greater than 3 and less than 5, the candidate restriction level may be 2.

In some embodiments, to ensure the diversity of the initial restriction schemes, the same candidate restriction region may generate the plurality of candidate restriction levels. For example, the restriction level determined based on the urban pollution degree may be floated up and down (e.g., plusing or minusing 1 level) to determine the plurality of candidate restriction levels.

In some embodiments, the candidate restriction level may also be related to traffic flow, that is, when determining the candidate restriction level, traffic flow may be used as a reference factor. The greater the traffic flow is, the greater the traffic impact caused by the restriction is, indicating that the traffic restriction scheme with a lower level may be used. For example, when the urban pollution degree is same, the higher the traffic flow is, the lower the candidate restriction level is. For another example, when the traffic flow is same, the higher the urban pollution degree is, the higher the candidate restriction level is.

In some embodiments, to ensure the diversity of the initial restriction scheme, the initial restriction level of each candidate restriction region may be directly and randomly determined. That is, the restriction levels may be randomly generated for the candidate restriction regions where the restriction measures may be implemented and used as the corresponding candidate restriction levels.

The initial restriction scheme may refer to the restriction scheme that takes the candidate restriction region as the execution region and the candidate restriction level as the restriction measure. In some embodiments, the restriction scheme may be presented in the form of a vector, that is, each region of the city may be coded first, and the current level of each region may be taken as the element value of the vector in turn according to the coding order. The elements of the vector may correspond to the various regions of the city, and the corresponding element value may reflect the restriction level of the region. In some embodiments, the plurality of initial restriction schemes may be generated correspondingly for regions with the plurality of restriction levels, and each initial restriction scheme corresponds to different restriction levels.

Step 224, determining the optimal restriction scheme through processing the plurality of initial restriction schemes based on the preset algorithm.

The preset algorithm may be an algorithm that may optimize the initial restriction scheme. That is, the preset algorithm may optimize the initial restriction scheme to determine the optimal restriction scheme. For example, the preset algorithm may include machine learning algorithms, which may process the initial restriction scheme based on machine learning algorithms to output the optimal restriction scheme.

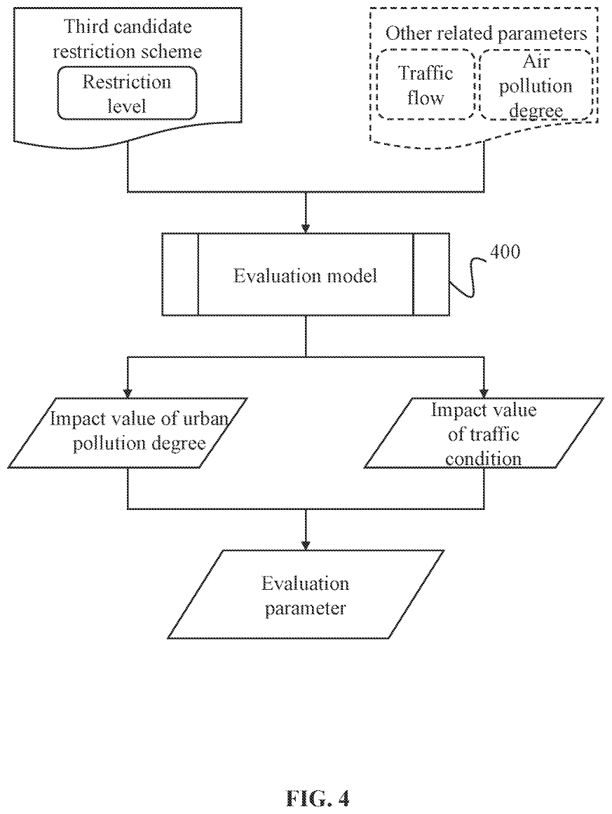

In some embodiments, the processing of a plurality of initial restriction schemes based on a preset algorithm may be iterative processing, that is, at least one round of iterative processing may be performed on the plurality of initial restriction schemes based on the preset algorithm to determine the optimal restriction scheme. Each round of iterative processing may determine at least one new restriction scheme, and the optimal restriction scheme may be determined based on the plurality of restriction schemes after each round of processing. For more content about iterative processing, please refer to FIG. 4 and its related descriptions.

Step 225, sending the optimal restriction scheme to the service platform and forwarding the optimal restriction scheme to the user platform.

In some embodiments, after the management platform determines the optimal restriction scheme, the management platform may package the data according to the optimal restriction scheme and send the packaged data to the service platform for storage. The service platform may send the corresponding optimal restriction scheme to the user platform as needed. For example, the management platform may generate the optimal restriction scheme for each future time period (such as holidays, major events, etc.) in advance according to the historical data, and store the optimal restriction scheme in the service platform in advance, and update the optimal restriction scheme in real-time. When the user needs to call the corresponding restriction scheme (such as near holidays), the calling instruction may be generated and sent to the service platform, and the service platform may call the optimal restriction scheme in the service platform corresponding to the calling instruction in response to the calling instruction and send the optimal restriction scheme to the user platform for presenting the optimal restriction scheme to the user.

The restriction scheme in a smart city provided in the present disclosure may determine the urban pollution degree of the city based on the urban pollution information so as to determine the optimal restriction scheme and reduce the influence of vehicle exhaust on urban air by implementing the restriction scheme, thereby reducing urban pollution.

FIG. 3 is an exemplary flowchart of determining an optimal restriction scheme according to some embodiments of the present disclosure. In some embodiments, process 300 may be executed by the management platform. The process 300 may describe the iterative process of the Nth round in iterative processing.

As shown in FIG. 3, process 300 includes the following steps.

Step 310, obtaining the first candidate restriction scheme of the current round and evaluation parameter of the first candidate restriction scheme of the current round.

The first candidate restriction scheme may be the initial candidate restriction scheme in each round of iteration. That is, in each round of iteration, the first candidate restriction scheme may be iterated as the initial value the round of the iteration for iteration processing. The presentation form of the first candidate restriction scheme may be consistent with that of the initial restriction scheme, and the first candidate restriction scheme may be presented as a vector containing the restriction levels of each region.

In some embodiments, the first candidate restriction scheme may be determined according to the results of the previous round of iteration processing. In some embodiments, for the first round of iteration (i.e., N=1), the first candidate restriction scheme may be the initial restriction scheme determined in step 223. For other rounds of iteration (i.e., N>1), the first candidate restriction scheme of current round may be determined according to the second candidate restriction scheme of the previous round and the third candidate restriction scheme of the previous round. The second candidate restriction scheme and the third candidate restriction scheme may be the processing results of each iteration, and the related content of the second candidate restriction scheme in each iteration may be found in the related description of step 320. For more contents of the third candidate restriction scheme, please refer to step 330 and its related descriptions.

Evaluation parameters may reflect the impact on traffic conditions after the implementation of the candidate restriction scheme. The higher the evaluation parameter is, the better the effect of the traffic restriction scheme is, that is, the better the positive impact on traffic conditions after the implementation of the restriction scheme is (such as significantly improving the congestion time). In some embodiments, the evaluation parameter may also reflect the impact on air pollution after the implementation of the candidate restriction scheme. For example, when the traffic condition is unchanged, the greater the evaluation parameter is, the greater the degree of purification of air pollution after the implementation of restriction scheme is.

In some embodiments, the evaluation parameter may be characterized as the weighted sum of the changes in urban traffic conditions (such as the reduction values of parameters such as average congestion time, the number of congested road sections, average congestion length, etc.) and the changes of urban pollution caused by vehicles (such as the reduction value of total exhaust emissions) after the implementation of the restriction scheme.

In some embodiments, the evaluation parameter of the first candidate restriction scheme may be obtained according to the current round of iteration. For the first round of iteration, the first candidate restriction scheme (i.e., the initial restriction scheme) may be processed according to the preset algorithm to determine the evaluation parameter of the first candidate restriction scheme. For other rounds of iterations, considering that the first candidate restriction scheme of current round may be the second candidate restriction scheme or the third candidate restriction scheme of the previous round, the evaluation parameters of the second candidate restriction scheme or the third candidate restriction scheme of the previous round have been determined in the previous round of iteration, then the corresponding evaluation parameters may be directly called as the evaluation parameters of the first candidate restriction scheme of current round.

In some embodiments, the urban traffic situation and urban pollution situation after the implementation restriction scheme may be estimated to determine the evaluation parameter. For more contents about determining the evaluation parameters, please refer to FIG. 5 and its related descriptions.

Step 320, determining the second candidate restriction scheme of current round from the first candidate restriction scheme of current round according to the evaluation parameter of the first candidate restriction scheme of current round.

The second candidate restriction scheme may be at least part of the first candidate restriction scheme with a better restriction effect. For example, the second candidate restriction scheme may be a restriction scheme of which the evaluation parameter is higher than the threshold or with the top evaluation parameters in the first candidate restriction scheme.

In some embodiments, for each of a plurality of first candidate restriction schemes, a selection parameter of the first candidate restriction scheme may be determined based on the evaluation parameter corresponding to the first candidate restriction scheme, and the selection parameter is used to characterize the initial probability that the first candidate restriction scheme is determined as the second candidate restriction scheme. The larger the selection parameter is, the higher the probability that the first candidate restriction scheme is determined as the second candidate restriction scheme is. For example, the selection parameter of the first candidate restriction scheme may be determined based on the ratio of the evaluation parameter corresponding to the first candidate restriction scheme to the sum of the evaluation parameters of all the first candidate restriction schemes.

In some embodiments, a plurality of second candidate restriction schemes may be determined from a plurality of first candidate restriction schemes based on the selection parameters corresponding to each of the plurality of first candidate restriction schemes. For example, the first candidate restriction scheme whose selection parameter is larger than the preset selection parameter threshold may be determined as the second candidate restriction scheme.

Step 330, determining the third candidate restriction scheme of current round through performing transforming processing on the second candidate restriction scheme of current round.

The third candidate restriction scheme may be a restriction scheme determined through at least partially modifying the restriction level of the second candidate restriction scheme. For example, at least part of the restriction region and restriction level of the second candidate restriction scheme may be adjusted to determine the third candidate restriction scheme.

In some embodiments, the transforming processing may refer to the change rule for the restriction region and the restriction level of the second candidate restriction scheme. For example, the transforming processing may include random changes, that is, transforming the current level of any region in the second candidate restriction scheme.

In some embodiments, the transforming processing may include the first transforming processing and the second transforming processing. In some embodiments, the first transforming processing and the second transforming processing are randomly implemented in proportion. For example, in 100 times of transforming processing, the second transforming processing may be no more than 5 times.

The first transforming processing may include exchanging the restriction level of the same candidate restriction region among a plurality of second candidate restriction schemes to generate a plurality of third candidate restriction schemes. For example, if the second candidate restriction schemes are (1, 1, 2, 3) and (1, 2, 1, 3) respectively, the first transforming processing may exchange the restriction level of the third region, and then the exchanged third candidate restriction schemes may be (1, 1, 1, 3) and (1, 2, 2, 3).

The second transforming processing includes adjusting the restriction level of one or more candidate restriction regions among a plurality of second candidate restriction schemes to generate a plurality of third candidate restriction schemes. For example, if the second candidate restriction scheme may be (1, 1, 2, 3), the second transforming processing may exchange the restriction levels of the second region and the restriction levels of the third region, and then the exchanged third candidate restriction scheme may be (1, 2, 1, 3).

In some embodiments, in the second candidate restriction scheme, the candidate restriction levels corresponding to the regions with high initial urban pollution degree may be exchanged or adjusted preferentially, so as to reduce the total urban pollution degree of the second candidate restriction scheme. Some embodiments of the present disclosure may improve the efficiency of determining the optimal restriction scheme by changing the candidate restriction level corresponding to the region with a high pollution degree in the second candidate restriction scheme.

In some embodiments, after the transforming processing is completed, the evaluation parameters of each second candidate restriction scheme and the third candidate restriction scheme may be determined, and the evaluation parameters of a plurality of second candidate restriction schemes and third candidate restriction schemes may be sorted in descending order, so as to eliminate the second candidate restriction schemes and/or the third candidate restriction schemes whose rank of evaluation parameters is below the preset ranking threshold.

Step 340, repeating the iterative processing until the preset condition is satisfied.

The preset condition may refer to the judgment condition for the completion of the iteration processing. That is, when the preset condition is satisfied, the iterative processing may be stopped and the following operations (e.g., step 350) may be performed. When the preset condition is not satisfied, the number of rounds of the current iteration may be increased by 1 (i.e., N+1) to execute the next round of iteration until the preset condition is satisfied.

In some embodiments, the preset condition may include at least one of time of iteration exceeding the threshold, convergence of evaluation parameter, and evaluation parameter satisfying the evaluation parameter threshold.

In some embodiments, when the preset condition is that the time of iterations exceeds a threshold, after the current iteration is completed, it may be determined whether the times of round (i.e., N) of the current iteration is greater than or equal to the preset times of iterations threshold (e.g., 50 times). In response to a determination that the times of round of the current iteration is greater than or equal to the preset times of iterations threshold, the preset condition is satisfied, and the iteration may be stopped. In response to a determination that the times of round of the current iteration is less than the preset times of iterations threshold, the next round of iteration may be performed until the times of round of iterations are greater than the preset times of iterations threshold.

In some embodiments, when the preset condition is that the evaluation parameters converge, the difference between the maximum evaluation parameter determined in the current round of iteration and the maximum evaluation parameter determined in the previous rounds may be judged. If the maximum evaluation parameter remains unchanged in the plurality of iterations or the difference between the maximum evaluation parameters of two iterations is lower than a certain convergence threshold (e.g., 0.1), the preset condition is satisfied, and the iteration may be stopped. Otherwise, the iteration processing may be performed continuously.

In some embodiments, when the preset condition is that the evaluation parameter satisfies the threshold condition, it may be determined whether the maximum evaluation parameter of the third candidate restriction scheme in the current round of iteration satisfies the threshold condition (for example, the maximum evaluation parameter is higher than the preset evaluation parameter threshold). If the maximum evaluation parameter of the third candidate restriction scheme in the current round of iteration satisfies the threshold condition, the preset condition is satisfied, and the iteration may be stopped. Otherwise, the iteration processing may be performed continuously. Specifically, step 340 may be implemented through the following steps.

Step 341, determining the evaluation parameter of the third candidate restriction scheme of current round.

In some embodiments, the evaluation parameter of the third candidate restriction scheme may be determined based on an evaluation model. For more descriptions of the evaluation model, please refer to FIG. 4 and its related descriptions.

Step 342, determining whether the current round of iteration satisfies the preset condition according to the evaluation parameter of the third candidate restriction scheme of current round.

In step 342, the preset condition may be characterized as a numerical adjustment of rating parameters (such as rating parameter threshold). That is, when the maximum evaluation parameter of each third candidate restriction scheme of current round of iteration satisfy the preset condition (for example, the maximum evaluation parameter is greater than the evaluation parameter threshold), the current round of iteration satisfies the preset condition. Otherwise, the current round of iteration does not satisfy the preset condition.

Step 343, in response to a determination that the current round of iteration does not satisfy the preset condition, determining the first candidate restriction scheme of the next round according to the second candidate restriction scheme of the current round and the third candidate restriction scheme of the current round, and executing the next round of iterative processing. That is, when the current round of iteration does not satisfy the preset condition, the next round of iteration may be performed.

In some embodiments, the second candidate restriction scheme of the current round and the third candidate restriction scheme of the current round may be screened according to the evaluation parameter to determine the first candidate restriction scheme of the next round. That is, the evaluation parameter of the second candidate restriction scheme of the current round and the evaluation parameter of the third candidate restriction scheme of the current round may be obtained first. The second candidate restriction scheme and the third candidate restriction scheme of current round may be screened based on the evaluation parameter of the second candidate restriction scheme of current round and the evaluation parameter of the third candidate restriction scheme of current round, and the screened candidate restriction scheme may be taken as the first candidate restriction scheme of the next round.

Step 344, in response to a determination that the current round of iteration satisfy the preset condition, stopping the iteration. That is, when current round of the iteration satisfies the preset condition, the iteration may be stopped and the optimal restriction scheme may be determined (i.e., executing step 350).

Step 350, when the preset condition is satisfied, determining the optimal restriction scheme according to the third historical third candidate restriction scheme of the historical iteration.

In some embodiments, the optimal restriction scheme may be determined based on the evaluation parameters of each historical third candidate restriction scheme. That is, the evaluation parameter of the third candidate restriction scheme may be obtained first, and then the candidate restriction scheme with the lowest evaluation parameters may be determined as the optimal restriction scheme according to the evaluation parameters of each historical third candidate restriction scheme of historical iteration.

Based on the iterative processing method provided by some embodiments of the present disclosure, the second candidate restriction scheme may be processed to expand the candidate restriction scheme, and the optimal restriction scheme may be determined from each candidate restriction scheme based on evaluation parameters, thus improving the accuracy of the optimal restriction scheme.

FIG. 4 is an exemplary structural diagram of an evaluation model according to some embodiments of the present disclosure.

The evaluation model may be a trained machine learning model. The evaluation model may be used to estimate change of the urban pollution degree and change of traffic conditions after the implementation of the candidate restriction scheme. Then, the evaluation parameters of the restriction scheme may be determined based on the change of urban pollution degree and change of traffic conditions after the candidate restriction scheme is implemented.

As shown in FIG. 4, the third candidate restriction scheme of current round may be processed based on the evaluation model 400 to determine the impact values of urban pollution degree and impact values of traffic conditions corresponding to the third candidate restriction scheme of current round. The evaluation model 400 may be a trained machine learning model. For example, the evaluation model 400 may be a DNN model and a CNN model. The input of the evaluation model 400 may include the third candidate restriction scheme, and the output of the evaluation model 400 may include the impact values of urban pollution degree and the impact values of traffic conditions after the third candidate restriction scheme is implemented.

It should be noted that the evaluation model 400 may also process other candidate restriction schemes according to the actual situation to determine the evaluation parameter of the candidate restriction scheme. For example, in the first round of iteration, the evaluation model 400 may also process the initial candidate restriction scheme to determine the first candidate restriction scheme and its corresponding evaluation parameter.

The impact value of urban pollution degree may reflect the change of urban pollution degree after the implementation of the candidate restriction scheme, i.e., the difference between the urban pollution degree after implementing the candidate traffic restriction scheme and the urban pollution degree before implementing the traffic restriction scheme. The greater the impact value of urban pollution degree is, the higher the implementation effect of the restriction scheme is, and the higher the air quality of the city is. In some embodiments, the impact value of urban pollution degree may be characterized by the reduction value of total vehicle exhaust emissions. That is, the impact value of urban pollution degree may be the absolute value of the difference between the total vehicle exhaust emissions before (or without) the implementation of the restriction scheme and the total vehicle exhaust emissions after the implementation of the restriction scheme.

The impact value of traffic conditions may reflect the change of traffic condition after the execution candidate restriction scheme. The greater the impact value of the traffic condition is, the better the traffic condition in the city after the implementation of the restriction scheme is. In some embodiments, the impact value of urban pollution degree may be characterized by the reduction value of data such as average congestion time, the number of congested road sections, average traffic jam length, etc. For example, the impact value of urban pollution degree may be determined by processing (such as weighted summation) the change values of data such as average congestion time, the number of congested road sections, and the average length of traffic jams before and after the implementation of the restriction scheme.

In some embodiments, the impact value of urban pollution degree and the impact value of traffic conditions may be processed based on preset processing rule to determine the evaluation parameter of the candidate restriction scheme. For example, the evaluation parameters may be determined by weighting the related parameters of the impact value of urban pollution degree and the related parameters of impact value of traffic condition (such as the change value of data such as total vehicle exhaust emissions, the average congestion time, the number of congested road sections, the average traffic jam length).

In some embodiments, the input of the evaluation model 400 may also include the air pollution degree and traffic flow of each candidate restriction region. The air pollution degree may be the output of the pollution prediction model. Traffic flow may be related data such as the number and speed of vehicles on various roads in the city. In some embodiments, traffic flow may be obtained from the third-party platform (such as transportation departments).

In some embodiments, the evaluation model 400 may be trained based on multiple sets of training samples with labels. Specifically, the training sample with label is input into the evaluation model, and the parameters of the evaluation model are updated through training. In some embodiments, a set of training samples may include: traffic flow and air pollution degree after or before the restriction scheme is implemented in the city. In some embodiments, the label may be the change value of urban pollution degree and the change value of traffic conditions before and after the implementation of the restriction scheme.

In some embodiments, the obtaining mode of the label may be determined according to the historical data of the city. For example, the unexecuted restriction scheme may be obtained from historical data. In some embodiments, the model may be trained by various methods based on the above samples to update the model parameters. For example, training may be performed based on gradient descent method. In some embodiments, when the trained evaluation model satisfies the preset condition, the training may be stopped. The preset condition may be that the result of the loss function converges or is smaller than a preset threshold, or the like.

Based on the evaluation model provided by some embodiments of the present disclosure, the machine learning algorithm may reasonably predict the execution effect of each restriction scheme, which improves the rationality of evaluating candidate restriction schemes, and then more reasonably determines the optimal restriction scheme.

In some embodiments, considering the complexity of the actual traffic situation, the specific data of impact value of the traffic situation may not be directly output in the evaluation model, but the traffic feature vector of the traffic situation (such as the traffic flow of each region after the restriction) after the implementation of the traffic restriction scheme may be output, and then the clustering algorithm may be performed according to the historical data to determine the more accurate impact value of traffic situation.

In some embodiments, one or more cluster centers may be determined according to historical data before clustering, which may include the following steps.

The historical data of urban traffic condition may be obtained and the historical feature vector of each historical data may be correspondingly generated to determine a first historical detection data set. The first historical detection data set may include historical feature vector and corresponding historical urban traffic condition (such as data on average congestion time, number of congested road sections, average traffic jam length, etc.). The elements of historical feature vector may correspond to the historical urban traffic condition.

The first cluster center set may be determined based on the first historical detection data collection. The first cluster center set may include one or more cluster centers. The cluster center may represent the type of detection result. In some embodiments, the set of first historical detection vectors may be clustered by a clustering algorithm to determine the first cluster center set. Clustering algorithms may include, but are not limited to, K-means clustering and/or density-based clustering (DBSCAN).

After determining one or more cluster centers, the feature vector of the restriction scheme may be compared with the clustering results to determine the cluster center, so as to determine the impact value of the traffic condition, which may include the following steps.

A first vector corresponding to the first detection data set may be determined based on the first detection data set. A first target cluster center may be determined based on the traffic feature vector and the first cluster center set after implementing the traffic restriction scheme. The first target cluster center may refer to the cluster center in the first cluster center set that is closest to the traffic feature vector (i.e., the distance between the cluster center in the first cluster center set the traffic feature vector is minimum) after the traffic restriction scheme is implemented. The method of calculating the distance may include Euclidean distance, cosine distance, Markov distance, Chebyshev distance, Manhattan distance, or the like, or any combination thereof.

After determining the first target cluster center, the average traffic condition of the cluster center may be used as the traffic condition after the implementation of the restriction scheme, and compared with the traffic condition before the implementation of the restriction scheme to determine the impact value of traffic condition after the implementation of the restriction scheme, so as to calculate the evaluation parameter of the candidate restriction scheme.

The basic concept has been described above. Obviously, for the technicians of the arts, the above-mentioned detailed disclosure is only used as an example, and does not constitute a limitation of the present disclosure. Although not explicitly described herein, various modifications, improvements, and corrections to this present disclosure may occur to those skilled in the art. Such modifications, improvements, and corrections are suggested in the present disclosure, so such modifications, improvements, and corrections still belong to the spirit and scope of the exemplary embodiments of the present disclosure.

At the same time, the present disclosure uses specific words to describe the embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. Further, certain features, structures, or features of one or more embodiments of the present disclosure may be combined.

Furthermore, unless explicitly stated in the claims, the order of processing elements and sequences described in this present disclosure, the use of alphanumeric, or the use of other names is not intended to limit the order of the processes and methods of this present disclosure. Although the above disclosure discusses through various examples what is currently considered to be a variety of useful embodiments of the disclosure, it is to be understood that such detail is solely for that purpose of description and that the appended claims are not limited to the disclosed embodiments, on the contrary, are intended to cover modifications and equivalent combinations that are within the spirit and scope of the embodiments of the present disclosure. For example, although the implementation of various components described above may be embodied in a hardware device, it may also be implemented as a software only solution, e.g., an installation on an existing server or mobile device.

Similarly, it should be noted that to simplify the expressions disclosed in the present disclosure and thus help the understanding of one or more embodiments of the invention, in the foregoing description of the embodiments of this specification, various features may sometimes be combined into one embodiment, drawings or descriptions thereof. However, this disclosure does not mean that object of the present disclosure requires more features than the features mentioned in the claims. Rather, claimed subject matter may lie in less than all features of a single foregoing disclosed embodiment.

Some embodiments use numbers that describe the number of components and attributes. It should be understood that such numbers used to describe the embodiments are modified by the modifiers “approximately”, “approximately” or “generally” in some examples. Unless otherwise stated, “about”, “approximately”, or “substantially” indicates that the number is allowed to vary by ±20%. Correspondingly, in some embodiments, the numerical parameters used in the specification and claims are approximate values, and the approximate values may be changed according to characteristics required by individual embodiments. In some embodiments, the numerical parameter should consider the prescribed effective digits and adopt a general digit retention method. Notwithstanding that the numerical ranges and parameters setting forth the broad scope of some embodiments of the application are approximations, the numerical values set forth in the specific examples are reported as precisely as practicable.

For each patent, patent application, patent application disclosure and other materials cited for this description, such as articles, books, descriptions, publications, documents, etc., the entire contents are hereby incorporated into this description for reference. The application history documents that are inconsistent or conflict with the content of the present disclosure are excluded, and the documents that restrict the broadest scope of the claims of the present disclosure (currently or later attached to the present disclosure) are also excluded. It should be noted that if the description, definition, and/or terms used in the appended materials of the present disclosure is inconsistent or conflicts with the content described in the present disclosure, the use of the description, definition and/or terms of the present disclosure shall prevail.

Finally, it should be understood that the embodiments described herein are only used to illustrate the principles of the embodiments of the present specification. Other modifications that may be employed may be within the scope of the application. Therefore, merely by way of example and not limitation, alternative configurations of the embodiments of the present disclosure may be considered consistent with the teachings of the present disclosure. Accordingly, embodiments of the present disclosure are not limited to that precisely as shown and described.

What is claimed is:

1. A method for determining a restriction scheme in a smart city, implemented based on an Internet of Things system for determining the restriction scheme in the smart city, wherein the Internet of Things system for determining the restriction scheme in the smart city includes a user platform, a service platform, a management platform, an object platform, and a sensor network platform, comprising:
determining urban pollution information of a city through the object platform, and sending the urban pollution information to the management platform through the sensor network platform, wherein the urban pollution information includes at least one of urban air image, air quality information, and vehicle exhaust information;
determining an optimal restriction scheme of the city according to the urban pollution information through the management platform, and sending the optimal restriction scheme to the user platform through the service platform, including:
determining an urban pollution degree of the city according to the urban pollution information;
determining at least one candidate restriction region according to the urban pollution degree;
determining a candidate restriction level of the at least one candidate restriction region, and generating a plurality of initial restriction schemes;
determining the optimal restriction scheme through processing the plurality of initial restriction schemes based on a preset algorithm; and
sending the optimal restriction scheme to the service platform and forwarding the optimal restriction scheme to the user platform.

2. The method of claim 1, wherein the determining an urban pollution degree of the city according to the urban pollution information includes:
determining the urban pollution degree of the city through processing the urban pollution information based on a pollution prediction model, wherein the pollution prediction model is a machine learning model.

3. The method of claim 1, wherein the management platform includes a plurality of management sub-platforms corresponding to each region of the city and a general database communicating with the plurality of management sub-platforms, and each management sub-platform is used to process the urban pollution information of a corresponding region, the general database is used to summarize regional data processed by each management sub-platform, and the regional data includes the urban pollution degree of the corresponding region.

4. The method of claim 1, wherein the determining the optimal restriction scheme through processing the plurality of initial restriction schemes based on a preset algorithm includes:
determining the optimal restriction scheme by performing at least one round of iterative processing on the plurality of initial restriction schemes based on the preset algorithm.

5. The method of claim 4, wherein the determining the optimal restriction scheme by performing at least one round of iterative processing on the plurality of initial restriction schemes based on the preset algorithm includes:
for Nth round of iterative processing, obtaining a first candidate restriction scheme of a current round and an evaluation parameter of the first candidate restriction scheme of the current round, wherein when N=1, the first candidate restriction scheme of the current round is determined according to the plurality of initial restriction schemes, when N>1, the first candidate restriction scheme is determined according to a second candidate restriction scheme of a previous round or a third candidate restriction scheme of the previous round;
determining the second candidate restriction scheme of the current round from the first candidate restriction scheme of the current round according to the evaluation parameter of the first candidate restriction scheme of the current round;
determining the third candidate restriction scheme of the current round through performing transformation processing on the second candidate restriction scheme of the current round;
repeating the iterative processing until a preset condition is satisfied; and
when the preset condition is satisfied, determining the optimal restriction scheme according to each historical third candidate restriction scheme of historical iterations.

6. The method of claim 5, wherein the repeating the iterative processing until a preset condition is satisfied includes:
for each round of iterative processing, determining the evaluation parameter of the third candidate restriction scheme of the current round;
determining whether the current iteration meets the preset condition according to the evaluation parameter of the third candidate restriction scheme of the current round;
in response to a determination that the current iteration meets the preset condition, stopping iteration; and
in response to a determination that the current iteration does not meet the preset condition, determining the first candidate restriction scheme of a next round according to the second candidate restriction scheme of the current round and the third candidate restriction scheme of the current round, and performing the next round of iterative processing.

7. The method of claim 6, wherein the determining the first candidate restriction scheme of a next round according to the second candidate restriction scheme of the current round and the third candidate restriction scheme of the current round, and performing the next round of iterative processing includes:
obtaining the evaluation parameter of the second candidate restriction scheme of the current round and the evaluation parameter of the third candidate restriction scheme of the current round; and
screening the second candidate restriction scheme of the current round and the third candidate restriction scheme of the current round based on the evaluation parameter of the second candidate restriction scheme of the current round and the evaluation parameter of the third candidate restriction scheme of the current round, and using the screened candidate restriction scheme as the first candidate restriction scheme of the next round.

8. The method of claim 6, wherein the determining evaluation parameter of the third candidate restriction scheme of the current round includes:
determining an urban pollution degree impact value and a traffic condition impact value corresponding to the third candidate restriction scheme of the current round through processing the third candidate restriction scheme of the current round based on an evaluation model, wherein the evaluation model is a machine learning model; and
determining the evaluation parameter of the third candidate restriction scheme based on the urban pollution degree impact value and the traffic condition impact value corresponding to the third candidate restriction scheme.

9. The method of claim 5, wherein the determining the optimal restriction scheme according to each historical third candidate restriction scheme of historical iterations includes:
obtaining the evaluation parameters of the historical third candidate restriction schemes; and
determining the optimal restriction scheme from the historical third candidate restriction schemes according to the evaluation parameters of the historical third candidate restriction schemes.

10. The method of claim 5, wherein the preset condition includes at least one of a number of iterations exceeding a threshold, convergence of the evaluation parameter, and the evaluation parameter satisfying an evaluation parameter threshold.

11. An Internet of Things system for determining a restriction scheme in a smart city, wherein the Internet of Things system for determining the restriction in the smart city includes a user platform, a service platform, a management platform, an object platform, and a sensor network platform;
the object platform is configured to determine urban pollution information of a city, and send the urban pollution information to the management platform through the sensor network platform, wherein the urban pollution information includes at least one of urban air image, air quality information, and vehicle exhaust information;
the management platform is configured to determine an optimal restriction scheme of the city according to the urban pollution information, and send the optimal restriction scheme to the user platform through the service platform;
wherein to determine an optimal restriction scheme of the city according to the urban pollution information and send the optimal restriction scheme to the user platform through the service platform, the management platform is further configured to:
determine an urban pollution degree of the city according to the urban pollution information;
determine at least one candidate restriction region according to the urban pollution degree;
determine a candidate restriction level of the at least one candidate restriction region, and generate a plurality of initial restriction schemes;
determine the optimal restriction scheme through processing the plurality of initial restriction schemes based on a preset algorithm; and
send the optimal restriction scheme to the service platform and forward the optimal restriction scheme to the user platform.

12. The system of claim 11, wherein the object platform is further configured to:
determine the urban pollution degree of the city through processing the urban pollution information based on a pollution prediction model, wherein the pollution prediction model is a machine learning model.

13. The system of claim 11, wherein the management platform includes a plurality of management sub-platforms corresponding to each region of the city and a general database communicating with the plurality of management sub-platforms, and each management sub-platform is configured to process the urban pollution information of a corresponding region, the general database is configured to summarize regional data processed by each management sub-platform, and the regional data includes the urban pollution degree of the corresponding region.

14. The system of claim 11, wherein the management platform is further configured to:
determine the optimal restriction scheme by performing at least one round of iterative processing on the plurality of initial restriction schemes based on the preset algorithm.

15. The system of claim 14, wherein the management platform is further configured to:
for Nth round of iterative processing, obtain a first candidate restriction scheme of a current round and an evaluation parameter of the first candidate restriction scheme of the current round, wherein when N=1, the first candidate restriction scheme of the current round is determined according to the plurality of initial restriction schemes, when N>1, the first candidate restriction scheme is determined according to a second candidate restriction scheme of a previous round or a third candidate restriction scheme of the previous round;
determine the second candidate restriction scheme of the current round from the first candidate restriction scheme of the current round according to the evaluation parameter of the first candidate restriction scheme of the current round;
determine the third candidate restriction scheme of the current round through performing transformation processing on the second candidate restriction scheme of the current round;
repeat the iterative processing until a preset condition is satisfied; and
when the preset condition is satisfied, determine the optimal restriction scheme according to each historical third candidate restriction scheme of historical iterations.

16. The system of claim 15, wherein the management platform is further configured to:
for each round of iterative processing, determine the evaluation parameter of the third candidate restriction scheme of the current round;
determine whether the current iteration meets the preset condition according to the evaluation parameter of the third candidate restriction scheme of the current round;
in response to a determination that the current iteration meets the preset condition, stop iteration; and
in response to a determination that the current iteration does not meet the preset condition, determine the first candidate restriction scheme of a next round according to the second candidate restriction scheme of the current round and the third candidate restriction scheme of the current round, and perform the next round of iterative processing.

17. The system of claim 16, wherein the management platform is further configured to:
obtain the evaluation parameter of the second candidate restriction scheme of the current round and the evaluation parameter of the third candidate restriction scheme of the current round; and
screen the second candidate restriction scheme of the current round and the third candidate restriction scheme of the current round based on the evaluation parameters of the second candidate restriction scheme of the current round and the evaluation parameters of the third candidate restriction scheme of the current round, and use the screened candidate restriction scheme as the first candidate restriction scheme of the next round.

18. The system of claim 16, wherein the management platform is further configured to:
determine an urban pollution degree impact value and a traffic condition impact value corresponding to the third candidate restriction scheme of the current round through processing the third candidate restriction scheme of the current round based on an evaluation model, wherein the evaluation model is a machine learning model; and
determine the evaluation parameter of the third candidate restriction scheme based on the urban pollution degree impact value and the traffic condition impact value corresponding to the third candidate restriction scheme.

19. The system of claim 15, wherein the management platform is further configured to:
obtain the evaluation parameters of the historical third candidate restriction schemes; and
determine the optimal restriction scheme from the historical third candidate restriction schemes according to the evaluation parameters of the historical third candidate restriction schemes.

20. A non-transitory computer-readable storage medium, wherein the storage medium stores computer instructions, when executed by a processor, the computer implements the method of claim 1.
